(19) United States
(12) Patent Application Publication
(10) Pub. No.: US 2026/0294375 A1
(43) Pub. Date: Oct. 1, 2026

(54) X-RAY CT APPARATUS, MEDICAL IMAGE PROCESSING APPARATUS, METHOD, AND STORAGE MEDIUM

(71) Applicant: CANON MEDICAL SYSTEMS CORPORATION, Tochigi (JP)

(72) Inventors: Junichi OOIDA, Ota-ku (JP); Hironaga NOGUCHI, Saitama (JP); Gakuto AOYAMA, Kawasaki (JP); Fumimasa SHIGE, Kawasaki (JP)

(73) Assignee: CANON MEDICAL SYSTEMS CORPORATION, Tochigi (JP)

(21) Appl. No.: 19/570,733

(22) Filed: Mar. 18, 2026

(30) Foreign Application Priority Data
Mar. 25, 2025 (JP) 2025-050336
Feb. 9, 2026 (JP) 2026-019299

(51) Int. Cl. A61B 6/50; A61B 6/00; A61B 6/03; G06T 7/00; G06T 7/11; G06T 7/246; G06V 10/25

(52) U.S. Cl. CPC A61B 6/507; A61B 6/032; A61B 6/503; A61B 6/5217; G06T 7/0012; G06T 7/11; G06T 7/246; G06V 10/25; G06T 2207/10081; G06T 2207/30048; G06T 2207/30104; G06V 2201/031

(57) ABSTRACT

An X-ray CT apparatus according to an embodiment includes processing circuitry that images a subject and acquires an X-ray CT image, acquires a region of interest based on an anatomical structure depicted in the X-ray CT image, acquires fluid information indicating a blood flow related to the region of interest and segments the region of interest into a plurality of regions based on the fluid information, and estimates pressure in the plurality of regions.

### CROSS-REFERENCE TO RELATED APPLICATIONS

This application is based upon and claims the benefit of priority from Japanese Patent Application No. 2025-050336, filed on Mar. 25, 2025; and Japanese Patent Application No. 2026-019299, filed on Feb. 9, 2026, the entire contents of which are incorporated herein by reference.

### FIELD

Embodiments described herein relate generally to an X-ray CT apparatus, a medical image processing apparatus, a method, and a storage medium.

### BACKGROUND

In diagnosis and treatment on the cardiovascular system, pressure applied to various structures in the heart and blood vessels is important information for the diagnosis and treatment. For example, if the pressure applied to the mitral valve can be grasped, it may be possible to predict rupture or thickening of the mitral valve. To perform treatment on the cardiovascular system, various devices, such as artificial valves and stents, may be used. After the treatment to place the devices is performed, it is necessary to make them be able to withstand the pressure. In other words, the pressure is important information related to the treatment plan for the cardiovascular system.

It is difficult to actually measure the pressure in the heart and blood vessels. While there have been developed devices that actually measure the pressure, such as pressure wires, the body part in which such a device can be used is limited. In addition, the use of the device causes the load of inserting it into the subject's body. Therefore, the method for estimating the pressure based on medical images is useful in many situations.

### BRIEF DESCRIPTION OF THE DRAWINGS

FIG. 1 is a block diagram of an example of a medical image processing system according to a first embodiment;

FIG. 2 is a flowchart for explaining a series of processing performed by a medical image processing apparatus according to the first embodiment;

FIG. 3 is a diagram of an example of a region of interest according to the first embodiment;

FIG. 4 is a diagram of an example of a regurgitation region according to the first embodiment;

FIG. 5 is a diagram of an example of region segmentation according to the first embodiment;

FIG. 6 is a conceptual diagram of pressure generated near the regurgitation region according to the first embodiment;

FIG. 7 is a conceptual diagram of the pressure generated near the regurgitation region according to a second embodiment;

FIG. 8 is a diagram of an example of the region segmentation according to the second embodiment; and

FIG. 9 is a block diagram of an example of an X-ray CT apparatus 100 according to another embodiment.

### DETAILED DESCRIPTION

An X-ray CT apparatus according to an embodiment includes processing circuitry that images a subject and acquires an X-ray CT image, acquires a region of interest based on an anatomical structure depicted in the X-ray CT image, acquires fluid information indicating a blood flow related to the region of interest and segments the region of interest into a plurality of regions based on the fluid information, and estimates pressure in the plurality of regions.

A medical image processing apparatus according to an embodiment includes processing circuitry that acquires a region of interest based on an anatomical structure depicted in a medical image, acquires fluid information indicating a blood flow related to the region of interest and segments the region of interest into a plurality of regions based on the fluid information, and estimates pressure in the plurality of regions.

A method according to an embodiment includes acquiring a region of interest based on an anatomical structure depicted in a medical image, acquiring fluid information indicating a blood flow related to the region of interest and segmenting the region of interest into a plurality of regions based on the fluid information, and estimating pressure in the plurality of regions.

A non-transitory computer readable medium according to an embodiment includes instructions that cause a computer to execute acquiring a region of interest based on an anatomical structure depicted in a medical image, acquiring fluid information indicating a blood flow related to the region of interest and segmenting the region of interest into a plurality of regions based on the fluid information, and estimating pressure in the plurality of regions.

Exemplary embodiments of an X-ray CT apparatus, a medical image processing apparatus, a method, and a storage medium are described below in greater detail with reference to the accompanying drawings.

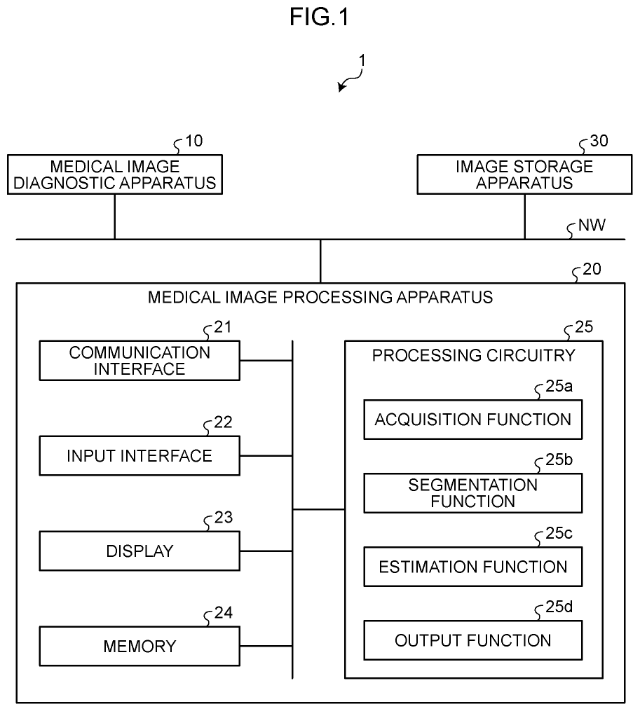

A first embodiment describes a medical image processing system 1 illustrated in FIG. 1 as an example. The medical image processing system 1 includes a medical image diagnostic apparatus 10, a medical image processing apparatus 20, and an image storage apparatus 30. The medical image diagnostic apparatus 10, the medical image processing apparatus 20, and the image storage apparatus 30 are communicably connected via a network NW.

The medical image diagnostic apparatus 10 is an apparatus that images a subject and acquires medical images. The medical image diagnostic apparatus 10 is a modality device, such as an X-ray computed tomography (CT) apparatus, an ultrasonic diagnostic apparatus, an X-ray diagnostic image, and a magnetic resonance imaging (MRI) apparatus. The medical image diagnostic apparatus 10 transmits the acquired medical images to the image storage apparatus 30 via the network NW.

The image storage apparatus 30 is an apparatus that stores therein various data acquired by the medical image diagnostic apparatus 10. For example, the image storage apparatus 30 stores therein various medical images acquired by the medical image diagnostic apparatus 10. The image storage apparatus 30 is, for example, a picture archiving and communication system (PACS) server.

While FIG. 1 illustrates one medical image diagnostic apparatus 10, the medical image processing system 1 may include a plurality of medical image diagnostic apparatuses 10. The medical image processing system 1 may include a plurality of types of medical image diagnostic apparatuses 10, such as an X-ray CT apparatus and an ultrasonic diagnostic apparatus.

The medical image processing apparatus 20 is an apparatus that analyzes the medical images acquired by the medical image diagnostic apparatus 10. As illustrated in FIG. 1, the medical image processing apparatus 20 includes, for example, a communication interface 21, an input interface 22, a display 23, a memory 24, and processing circuitry 25.

The communication interface 21 controls transmission and communications of various data transmitted to and received from other apparatuses or systems connected to the medical image processing apparatus 20 via the network NW. Specifically, the communication interface 21 is connected to the processing circuitry 25 and outputs data received from other apparatuses or systems to the processing circuitry 25 or transmits data output from the processing circuitry 25 to other apparatuses or systems. The communication interface 21 is implemented by, for example, a network card, a network adapter, or a network interface controller (NIC).

The input interface 22 receives various input operations from an operator of the medical image processing apparatus 20, converts the received input operation into electrical signals, and outputs them to the processing circuitry 25. The input interface 22 is implemented by, for example, a mouse, a keyboard, a trackball, a switch, a button, a joystick, a touchpad on which the operator performs an input operation by touching an operation surface, a touchscreen that integrates a display screen and a touchpad, a non-contact input circuit with an optical sensor, or a voice input circuit. The input interface 22 may be configured as a tablet terminal device or the like that can wirelessly communicate with the medical image processing apparatus 20 itself. The input interface 22 may be a circuit that receives the input operation from the operator by motion capture. For example, the input interface 22 can receive the operator's body movement, line of sight, or the like as the input operation by processing signals acquired via a tracker or images acquired about the operator. The input interface 22 is not limited only to those with physical operation components, such as a mouse and a keyboard. Examples of the input interface 22 also include an electrical signal processing circuit that receives electrical signals corresponding to an input operation from an external input device provided separately from the medical image processing apparatus 20 and outputs the electrical signals to the processing circuitry 25.

The display 23 displays various kinds of information. For example, the display 23 displays a graphical user interface (GUI) to receive various instructions and settings from the operator via the input interface 22. The display 23 is, for example, a liquid crystal display or a cathode ray tube (CRT) display. The display 23 may be a desktop display or may be configured as a tablet terminal device or the like that can wirelessly communicate with the medical image processing apparatus 20 itself.

The medical image processing apparatus 20 may include a projector instead of or in addition to the display 23. The projector can project images on a screen, wall, floor, or the like under the control of the processing circuitry 25. For example, the projector can project images on any desired plane, object, space, or the like by projection mapping.

The memory 24 is implemented by, for example, a semiconductor memory element, such as a random access memory (RAM) and a flash memory, a hard disk, or an optical disc. For example, the memory 24 stores therein medical images acquired by the medical image diagnostic apparatus 10 and various methods generated by the processing circuitry 25. The processing circuitry 25 stores therein computer programs for the circuits included in the medical image processing apparatus 20 to implement their functions. The memory 24 may be implemented by a group of servers (cloud) connected to the medical image processing apparatus 20 via the network NW.

The processing circuitry 25 includes an acquisition function 25a, a segmentation function 25b, an estimation function 25c, and an output function 25d. The acquisition function 25a is an example of an acquisition unit. The segmentation function 25b is an example of a segmentation unit. The estimation function 25c is an example of an estimation unit. The output function 25d is an example of an output unit.

For example, the processing circuitry 25 functions as the acquisition function 25a by reading a computer program corresponding to the acquisition function 25a from the memory 24 and executing it. Similarly, the processing circuitry 25 can function as the segmentation function 25b, the estimation function 25c, and the output function 25d. The acquisition function 25a, the segmentation function 25b, the estimation function 25c, and the output function 25d will be described later in greater detail.

In the medical image processing apparatus 20 illustrated in FIG. 1, each processing function is stored in the memory 24 as a computer-executable program. The processing circuitry 25 is a processor that reads each computer program from the memory 24 and executes it to implement the function corresponding to the computer program. In other words, the processing circuitry 25 that has read a computer program has the function corresponding to the read computer program.

In FIG. 1, a single processing circuitry 25 implements the acquisition function 25a, the segmentation function 25b, the estimation function 25c, and the output function 25d. Alternatively, a plurality of independent processors may be combined to constitute the processing circuitry 25, and each processor may execute the computer program to implement the corresponding function. The processing functions of the processing circuitry 25 may be distributed or integrated into a single or a plurality of processing circuits as appropriate.

The processing circuitry 25 may implement the functions using a processor of an external apparatus connected via the network NW. For example, the processing circuitry 25 implements the functions illustrated in FIG. 1 by reading the computer programs corresponding to the respective functions from the memory 24 and executing them, and using a group of servers (cloud) connected to the medical image processing apparatus 20 via the network NW as computing resources.

The network NW illustrated in FIG. 1 may be a local network closed in a hospital or a network via the Internet. For example, the medical image processing apparatus 20 may be installed in the same facility as the medical image diagnostic apparatus 10 and the image storage apparatus 30 or in different facilities.

The above has described the entire configuration of the medical image processing system 1 including the medical image diagnostic apparatus 10, the medical image processing apparatus 20, and the image storage apparatus 30. With the configuration described above, the medical image processing apparatus 20 can accurately estimate the pressure in the parts of the cardiovascular system, such as the heart and blood vessels, while reducing the processing load.

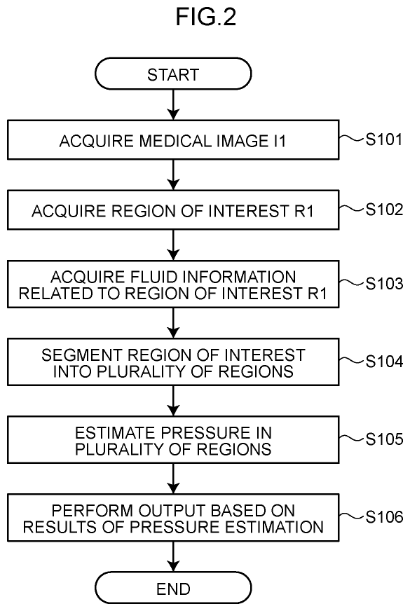

The following describes the embodiment in greater detail with reference to the flowchart in FIG. 2. FIG. 2 is a flowchart for explaining a series of processing performed by the medical image processing apparatus 20 according to the first embodiment.

Each step in FIG. 2 is performed by the processing circuitry 25 included in the medical image processing apparatus 20. Specifically, Steps S101 and S102 correspond to the acquisition function 25a. Steps S103 and S104 correspond to the segmentation function 25b. Step S105 corresponds to the estimation function 25c. Step S106 corresponds to the output function 25d.

First, the processing circuitry 25 acquires a medical image I1 obtained by imaging the subject (Step S101). While the type of the medical image I1 is not limited, examples of the medical image I1 include, but are not limited to, X-ray CT images, ultrasound images (echo images), X-ray images, such as angiograms, MR images, etc.

For example, the medical image diagnostic apparatus 10 images the subject to acquire the medical image I1 and transmits the acquired medical image I1 to the image storage apparatus 30. The image storage apparatus 30 manages the received medical image I1 on a system, such as PACS. The processing circuitry 25 can acquire the medical image I1 of the subject from the image storage apparatus 30 via the network NW. Alternatively, the processing circuitry 25 may acquire the medical image I1 directly from the medical image diagnostic apparatus 10 not via the image storage apparatus 30.

Subsequently, the processing circuitry 25 acquires a region of interest R1 based on an anatomical structure depicted in the medical image I1 (Step S102). The region of interest R1 is data that represents geometric characteristics of the part of interest in which a user, such as a doctor, is interested. The present embodiment uses the mitral valve as the part of interest, for example, and describes an example where the region corresponding to the mitral valve is acquired as the region of interest R1.

For example, the processing circuitry 25 can automatically acquire the region of interest R1 by image processing on the pixel value (e.g., luminance value) of each pixel constituting the medical image I1. To acquire the region of interest R1, the processing circuitry 25 may use a graph cut method, a mean shift method, or a deep learning (DL) method, such as U-net. For example, the processing circuitry 25 can acquire the region of interest R1 by the method disclosed in Non-patent Literature 1.

Alternatively, the processing circuitry 25 may acquire the region of interest R1 based on an operation performed by the user. For example, the processing circuitry 25 can acquire the region of interest R1 by displaying the medical image I1 on the display 23 and receiving an operation to select the region of interest R1.

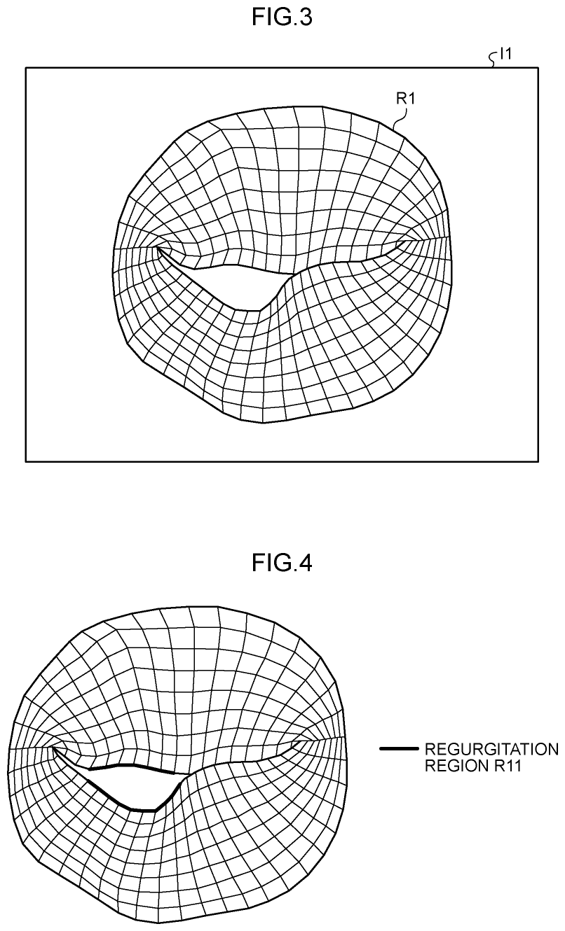

An example of representation of the region of interest R1 is a predetermined number of pieces of point cloud data constituting the surface of the region of interest R1. Alternatively, the region of interest R1 can be represented by, for example, a predetermined number of pieces of point cloud data constituting the region of interest R1 or mesh data having the point cloud data as nodes. The following describes the region of interest R1 as the mesh data illustrated in FIG. 3.

Subsequently, the processing circuitry 25 acquires fluid information related to the region of interest R1 (Step S103). For example, the processing circuitry 25 acquires regurgitation velocity distribution in the neighborhood of the region of interest R1 as the fluid information. The regurgitation velocity distribution can be acquired based on, for example, a Doppler image (Doppler data) serving as a type of ultrasound image.

Specifically, the medical image diagnostic apparatus 10 serving as an ultrasonic diagnostic apparatus first performs ultrasonic scanning on the subject using an ultrasonic probe. The ultrasonic probe is a device that uses piezoelectric transducer elements to generate ultrasonic waves and receives reflected waves to convert them into electrical signals. When ultrasonic waves are transmitted from the ultrasonic probe, the transmitted ultrasonic waves are reflected one after another on a discontinuity plane of acoustic impedance in the body tissue of the subject and are received as reflected wave signals by a plurality of piezoelectric transducer elements of the ultrasonic probe. The amplitude of the received reflected wave signals depends on the difference in acoustic impedance at the discontinuity plane on which the ultrasonic waves are reflected. When the transmitted ultrasonic pulses are reflected by a moving blood flow or a surface, such as the heart wall, the reflected wave signals depend on the velocity component of the moving object with respect to the direction of transmission of the ultrasonic waves due to the Doppler effect and are subjected to frequency deviation.

There are two types of ultrasonic probes: one-dimensional ultrasonic probes and two-dimensional ultrasonic probes. One-dimensional ultrasonic probes have a configuration in which a plurality of piezoelectric transducer elements are arranged in a row. Two-dimensional ultrasonic probes have a configuration in which a plurality of piezoelectric transducer elements are two-dimensionally arranged in a grid pattern. Alternatively, two-dimensional ultrasonic probes are implemented by mechanically oscillating a plurality of piezoelectric transducer elements of a one-dimensional ultrasonic probe.

For example, the processing circuitry included in the ultrasonic diagnostic apparatus performs logarithmic amplification, envelope detection, or other processing on the received reflected wave data to generate data (B-mode data) that represents the signal intensity at each sample point by the brightness of luminance. The processing circuitry included in the ultrasonic diagnostic apparatus also generates data (Doppler data) by extracting motion information of the moving object based on the Doppler effect at each sample point in the scanning region from the reflected wave data. Specifically, the processing circuitry performs frequency analysis on velocity information from the reflected wave data, extracts echo components of blood flows, tissues, and contrast agents due to the Doppler effect, and generates data (Doppler data) by extracting moving object information, such as average velocity, dispersion, and power, from a plurality of points. Examples of the moving object include, but are not limited to, blood flows, tissues, such as the heart wall, contrast agents, etc. When the scanning is performed using a one-dimensional ultrasonic probe, two-dimensional data is acquired as the B-mode data and the Doppler data. When the scanning is performed using a two-dimensional ultrasonic probe, three-dimensional data is acquired as the B-mode data and the Doppler data.

As described above, the medical image diagnostic apparatus 10 serving as an ultrasonic diagnostic apparatus can acquire three-dimensional Doppler data. The three-dimensional Doppler data provides fluid information on the blood included in the three-dimensional space subjected to ultrasonic scanning. The processing circuitry 25 can acquire, for example, the distribution of the flow velocity of the blood flow based on the three-dimensional Doppler data.

The processing circuitry 25 acquires, for example, the regurgitation velocity distribution of a predetermined phase in the heartbeat of a subject P. For example, an electrocardiogram of the subject is collected in synchronization with the ultrasonic scanning performed by the ultrasonic diagnostic apparatus. The processing circuitry 25 can acquire an electrocardiogram as incidental information of the three-dimensional Doppler data, for example, identify the three-dimensional Doppler data of a predetermined phase based on the electrocardiogram, and acquire the regurgitation velocity distribution of the predetermined phase. For example, the processing circuitry 25 acquires two “R waves” based on the waveform of the electrocardiogram and identifies the first half of the periods obtained by dividing the time interval between the two waves into two at “4:6” as the systole.

Subsequently, the processing circuitry 25 aligns the regurgitation velocity distribution of the predetermined phase with the region of interest R1. For example, the processing circuitry 25 first identifies the three-dimensional Doppler data at the phase closest to the mid-systole and aligns the identified three-dimensional Doppler data with the region of interest R1 acquired at Step S102 by various image processing techniques.

Subsequently, the processing circuitry 25 sets a region within a predetermined distance from the aligned region of interest R1 on the three-dimensional Doppler data and acquires the flow velocity information assigned to the pixels in the region as the fluid information related to the region of interest R1. The flow velocity information may include the direction besides the flow velocity value (speed).

The elements constituting image data may be distinguished as pixels in the case of two-dimensional data and voxels in the case of three-dimensional data. To simplify the explanation, they are not distinguished from each other, and both are denoted as pixels.

The image processing technique for alignment may be, for example, free-form deformation (FFD). Alternatively, large deformation diffeomorphic metric mapping (LDDMM) may be employed. Still alternatively, deep learning may be employed. For example, the processing circuitry 25 can perform alignment using reinforcement learning disclosed in Non-patent Literature 2 or other literatures.

The processing circuitry 25 may identify a regurgitation region R11 illustrated in FIG. 4 and acquire the fluid information related to the region of interest R1 based on the regurgitation region R11. For example, the processing circuitry 25 acquires the nodes belonging to the valve orifice of the anterior cusp from the mesh data acquired as the region of interest R1 and calculates the distance between each node and all the nodes belonging to the valve orifice of the posterior cusp. If the shortest distance is equal to or larger than a threshold, the processing circuitry 25 considers the region to be the regurgitation region R11. The processing circuitry 25 identifies the region where the shortest distance is, for example, “1 mm” or larger as the regurgitation region R11. Subsequently, the processing circuitry 25 sets a region within a predetermined distance from the regurgitation region R11 in the aligned region of interest R1 on the three-dimensional Doppler data at the phase closest to the mid-systole and acquires the flow velocity information assigned to the pixels in the region as the fluid information related to the region of interest R1.

While the above has described an example where the regurgitation velocity distribution at the predetermined phase is acquired as the fluid information related to the region of interest R1, the embodiment is not limited thereto. For example, the processing circuitry 25 may acquire, as the fluid information, the distribution of time-averaged regurgitation velocity in one beat, the distribution of maximum regurgitation velocity in one beat, the distribution of vorticity at the predetermined phase, the distribution of momentum at the predetermined phase, or the distribution of kinetic energy at the predetermined phase. In other words, the fluid information acquired at Step S103 is any kind of information indicating the blood flow.

The following describes a method for acquiring the distribution of time-averaged regurgitation velocity in one beat. For example, the processing circuitry 25 prepares segmented regions finely segmented at predetermined intervals with the center of the valve annulus of the aligned mitral valve in the systole described above at the origin. Subsequently, the processing circuitry 25 calculates the flow velocity value in the segmented region based on the distance between the segmented region and the pixel in the three-dimensional Doppler data and the fluid information assigned to the pixel at each phase. The processing circuitry 25 performs this processing at all the phases of the systole and calculates the average between the phases, thereby acquiring the distribution of time-averaged regurgitation velocity in one beat.

Subsequently, the processing circuitry 25 segments the region of interest R1 into a plurality of regions based on the fluid information acquired at Step S103 (Step S104). The following describes an example where the regurgitation velocity distribution is used as the fluid information.

First, the processing circuitry 25 acquires the flow velocity information corresponding to each node of the mesh data acquired as the region of interest R1. For example, the processing circuitry 25 acquires, for each node, the value of the pixel closest to the node in the regurgitation velocity distribution acquired at Step S103.

Alternatively, the processing circuitry 25 may acquire, for each node, a value based on the values of a plurality of pixels whose distance from the node is within a predetermined threshold in the regurgitation velocity distribution acquired at Step S103. For example, the processing circuitry 25 acquires statistics (mean, median, etc.) of the values of the pixels whose distance from the node is within the predetermined threshold. Alternatively, for example, the processing circuitry 25 acquires the largest value of the values of the pixels whose distance from the node is within the predetermined threshold. Still alternatively, for example, the processing circuitry 25 acquires the largest value of the values of the pixels whose distance from the node is within the predetermined threshold and whose direction of the flow velocity is within a predetermined range. If no pixel whose distance is within the threshold is present, the value of the node may be set to “0”.

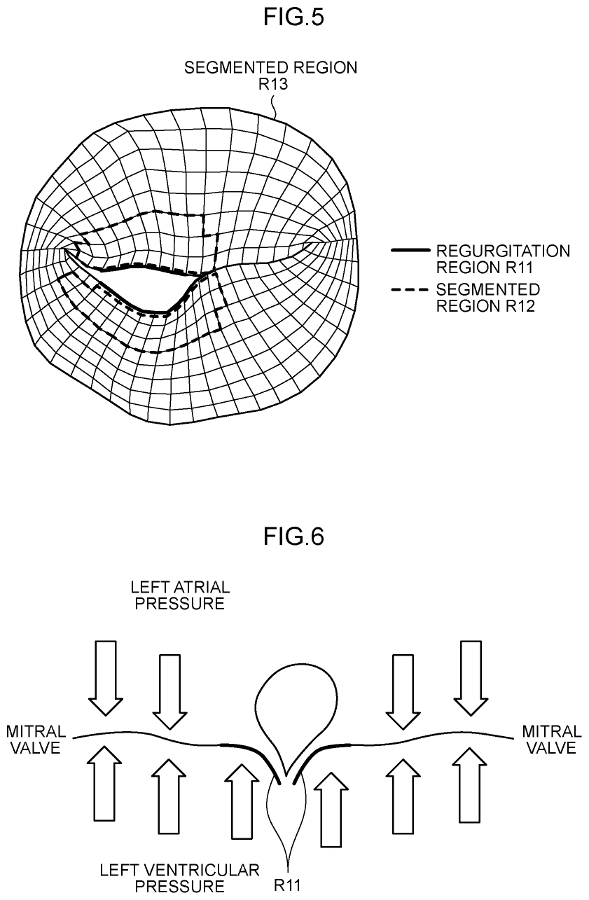

Subsequently, the processing circuitry 25 segments the region of interest R1 into a plurality of regions. For example, the processing circuitry 25 identifies the portion of the region of interest R1 where the flow velocity value exceeds a predetermined threshold as a segmented region R12 illustrated in FIG. 5 based on the flow velocity information corresponding to each node. The processing circuitry 25 identifies the portion of the region of interest R1 other than the segmented region R12 as a segmented region R13. In other words, the processing circuitry 25 segments the region of interest R1 into two regions of the segmented region R12 and the segmented region R13. The segmented region R12 is an example of a first segmented region. The segmented region R13 is an example of a second segmented region.

The properties of the segmented regions R12 and R13 are described with reference to FIG. 6. FIG. 6 is a conceptual diagram of pressure generated near the regurgitation region R11.

During the left ventricular systole, the mitral valve receives force due to the blood pressure from the left ventricle and the left atrium. When regurgitation occurs, however, force due to the blood flow is generated from the left ventricle to the left atrium near the regurgitant valve orifice. Therefore, in the regurgitation region R11 illustrated in FIG. 6, the mitral valve receives the pressure from the left ventricle, but the pressure from the left atrial pressure significantly decreases. Thus, the pressure received by the mitral valve from the left atrium differs between the part near the regurgitant valve orifice and the other part. The segmented region R12 segmented in the manner described above represents the region near the regurgitant valve orifice where the pressure from the left atrial pressure decreases. The segmented region R13 represents the other region where the pressure from the left atrial pressure does not decrease.

Subsequently, the processing circuitry 25 estimates the pressure in a plurality of regions (Step S105). In other words, the processing circuitry 25 estimates the pressure for each of the segmented regions R12 and R13.

For example, the processing circuitry 25 estimates the pressure in the entire mitral valve and then estimates the pressure in each segmented region. The processing circuitry 25 can estimate the pressure in the entire mitral valve by the method described in Non-patent Literature 3, for example. The processing circuitry 25 may measure the left ventricular pressure gradient using ultrasound images as disclosed in Non-patent Literature 4, for example.

As described with reference to FIG. 6, the segmented region R12 indicates the region near the regurgitant valve orifice where the pressure from the left atrial pressure decreases. Therefore, the processing circuitry 25 sets the pressure in the segmented region R12 to be smaller than the pressure in the segmented region R13 by a predetermined specified value. In other words, the processing circuitry 25 can set the following expression: “(Pressure in Segmented Region R12)=(Pressure in Segmented Region R13)−(Specified Value)”.

There is no particular limitation on the method for setting the specified value serving as the difference between the pressure in the segmented region R12 and that in the segmented region R13. For example, the specified value may be a literature value or may be set by the user. The processing circuitry 25 may set the specified value by, for example, obtaining the maximum value of the flow velocity based on the fluid information, dividing it by a reference value of the flow velocity obtained from a literature value or the like, and adding the pressure gradient obtained from a literature value or the like.

The processing circuitry 25 can also set the following expression: “(Pressure in Entire Mitral Valve)×(Area of Entire Region of Mitral Valve)=(Pressure in Segmented Region R12)×(Area of Segmented Region R12)+(Pressure in Segmented Region R13)×(Area of Segmented Region R13)”. The processing circuitry 25 can solve these two expressions to estimate the pressure in each of the segmented regions R12 and R13.

The processing circuitry 25 performs output based on the results of estimation of the pressure in a plurality of regions (Step S106). For example, the processing circuitry 25 displays the results of pressure estimation on the display 23. The processing circuitry 25, for example, may transmit the results of pressure estimation to a display apparatus (viewer) used for viewing medical information or to a terminal device of the user, such as a doctor.

Alternatively, the processing circuitry 25 may perform analysis based on the results of pressure estimation and output the analysis results. For example, the processing circuitry 25 may generate diagnostic support information, such as an evaluation of mitral valve disease in the subject, based on the results of pressure estimation and output the diagnostic support information.

The processing circuitry 25 may analyze the pressure distribution based on the results of pressure estimation and output the estimated pressure distribution. For example, the processing circuitry 25 estimates the pressure distribution by modeling the pressure applied to the mitral valve based on the pressure estimated for each of the segmented regions R12 and R13 and estimating parameters by data assimilation or other techniques.

Examples of the method for modeling the pressure include, but are not limited to, the method described in Non-patent Literature 5, the method described in Non-patent Literature 6, etc. However, when the pressure applied to the mitral valve is modeled by one parameter as described in Non-patent Literature 5, deviation may possibly occur between the model and the actual phenomenon, thereby affecting the accuracy in analysis of the pressure distribution. In the method of applying individual pressure to each mesh obtained by dividing the mitral valve as described in Non-patent Literature 6, the processing load increases because there are as many parameters related to the pressure as the number of meshes. By contrast, the pressure estimation performed by the processing circuitry 25 enables accurately estimating the pressure while reducing the processing load, and thus enables accurately estimating the pressure distribution by parameter optimization.

In FIG. 5, the region of interest R1 is segmented into the two regions of the segmented region R12 and the segmented region R13. However, the embodiment is not limited thereto, and the processing circuitry 25 may segment the region of interest R1 into three or more regions.

For example, the processing circuitry 25 may identify the segmented region R12 as illustrated in FIG. 5 and further segment the segmented region R13 into a plurality of regions. In other words, the processing circuitry 25 may identify a plurality of regions as the second segmented region.

For example, the processing circuitry 25 sets a first threshold as a predetermined threshold and also sets a second threshold smaller than the first threshold. The processing circuitry 25 identifies the portion of the region of interest R1 where the flow velocity value exceeds the first threshold as the segmented region R12 illustrated in FIG. 5. The processing circuitry 25 identifies the portion of the region of interest R1 other than the segmented region R12 as the segmented region R13. The processing circuitry 25 identifies the portion of the segmented region R13 where the flow velocity value exceeds the second threshold as a first sub-segmented region and identifies the portion of the segmented region R13 other than the first sub-segmented region as a second sub-segmented region.

For another example, the processing circuitry 25 identifies the portion of the segmented region R13 corresponding to the anterior cusp as the first sub-segmented region and identifies the portion corresponding to the posterior cusp as the second sub-segmented region.

As described above, the acquisition function 25a according to the first embodiment acquires the region of interest R1 based on the anatomical structure depicted in the medical image I1. The segmentation function 25b acquires the fluid information related to the region of interest R1 and segments the region of interest R1 into a plurality of regions based on the fluid information. The estimation function 25c estimates the pressure in the segmented regions. Thus, the medical image processing apparatus 20 can accurately estimate the pressure in the parts of the cardiovascular system while reducing the processing load.

The first embodiment above has described an example where the fluid information mainly in systole is used as the fluid information related to the region of interest R1. By contrast, a second embodiment describes a case where the flow velocity in diastole is used.

The medical image processing system 1 according to the second embodiment has the same configuration as the medical image processing system 1 illustrated in FIG. 1, and is different from it in the processing performed by the acquisition function 25a and the segmentation function 25b. In the following description, the points described in the first embodiment are denoted by the same reference numerals as in FIG. 1, and explanation thereof is omitted.

First, the processing circuitry 25 according to the second embodiment acquires the medical image I1 and the region of interest R1 as in the processing at Steps S101 and S102 in FIG. 2. Subsequently, the processing circuitry 25 acquires fluid information on the part near the mitral valve in the left ventricle in diastole as the fluid information at Step S103. Similarly to the first embodiment, the fluid information is any kind of information indicating the blood flow, such as flow velocity, vorticity, momentum, and kinetic energy. For example, the processing circuitry 25 can acquire the fluid information on the part near the mitral valve in the left ventricle in diastole based on three-dimensional Doppler data.

Alternatively, the processing circuitry 25 may acquire the fluid information on the part near the mitral valve in the left ventricle in diastole by fluid simulation. For example, the processing circuitry 25 can acquire the fluid information on the part near the mitral valve in the left ventricle in diastole by acquiring the temporal changes in the shape of the left ventricle and the blood inflow into the left ventricle in diastole based on the medical image I1, such as an X-ray CT image, acquired from the subject and performing a fluid simulation.

Alternatively, the processing circuitry 25 may acquire the fluid information on the part near the mitral valve in the left ventricle in diastole using a predetermined lookup table. For example, the processing circuitry 25 acquires in advance a plurality of results of calculation of the fluid information corresponding to the temporal changes in the shape of the left ventricle and the blood inflow into the left ventricle in diastole and creates a response surface. The processing circuitry 25 can acquire the fluid information by acquiring the temporal changes in the shape of the left ventricle and the blood inflow into the left ventricle in diastole based on the medical image I1 acquired from the subject and applying the acquired information to the response surface.

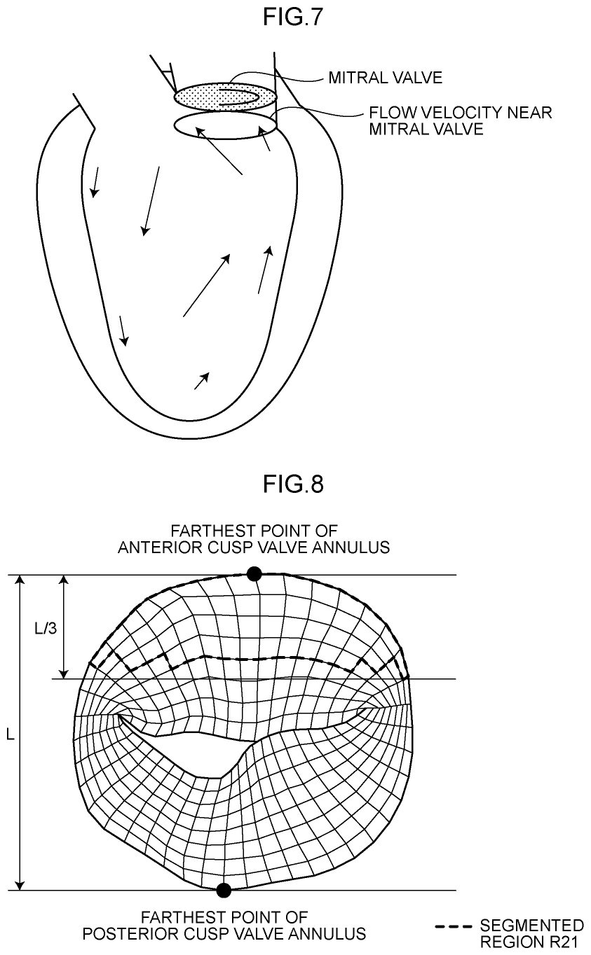

Subsequently, the processing circuitry 25 segments the region of interest R1 into a plurality of regions based on the fluid information on the part near the mitral valve in diastole as the processing at Step S104. In diastole, for example, a vortex is generated in the left ventricle as illustrated in FIG. 7, and force due to the vortex is applied to the mitral valve. The processing circuitry 25 estimates the magnitude of the force applied to the mitral valve by the vortex based on the fluid information on the part near the mitral valve and segments the region of interest R1 into a plurality of regions.

Specifically, FIG. 7 is a schematic of the left ventricle and illustrates the mitral valve as the blood inlet to the left ventricle. In FIG. 7, the aortic valve serving as the blood outlet from the left ventricle is positioned on the left of the mitral valve. It is known that the blood flow in the left ventricle swirls in diastole as indicated by the arrows in FIG. 7. Therefore, the part of the mitral valve on the aortic valve side (left side in FIG. 7) is subjected to force caused by the collision of the blood flow swirling in the left ventricle. From this perspective, the processing circuitry 25 segments the region of interest R1 into a plurality of regions.

For example, the processing circuitry 25 derives a plane perpendicular to a line connecting the center of the left ventricle and the center of the left atrium and projects the mitral valve on the plane. Subsequently, the processing circuitry 25 derives a line connecting two projected commissures and derives the farthest point of the anterior cusp valve annulus at which the distance between the point belonging to the valve annulus of the anterior cusp of the projected mitral valve and the line is the longest. The processing circuitry 25 performs the same operation on the points belonging to the valve annulus of the posterior cusp to derive the farthest point of the posterior cusp valve annulus. The farthest point of the anterior cusp valve annulus and the farthest point of the posterior cusp valve annulus are illustrated in FIG. 8. FIG. 8 is a diagram of an example of the region segmentation according to the second embodiment.

Subsequently, the processing circuitry 25 calculates the distance “L” between the farthest point of the anterior cusp valve annulus and the farthest point of the posterior cusp valve annulus. As illustrated in FIG. 8, the processing circuitry 25 acquires the region where the distance “L” from the farthest point of the anterior cusp valve annulus is at a specific ratio (e.g., “L/3”) as a segmented region R21. The processing circuitry 25 identifies the portion of the region of interest R1 other than the segmented region R21 as a segmented region R22. In other words, the processing circuitry 25 segments the region of interest R1 into two regions of the segmented region R21 and the segmented region R22.

While the specific ratio is “L/3” in FIG. 8, it can be modified as needed. For example, the processing circuitry 25 sets the specific ratio based on the flow velocity information on the part near the mitral valve in the left ventricle in diastole. The processing circuitry 25, for example, may calculate the average of the flow velocity of the flow velocity information and set the specific ratio to “L/3” when the average exceeds a threshold and to “L/4” otherwise. The processing circuitry 25, for example, may use a ratio obtained by acquiring the maximum value of the flow velocity of the flow velocity information, dividing it by the reference value of the flow velocity obtained from a literature value or the like, and dividing the obtained value by “4”.

As described above, the processing circuitry 25 according to the second embodiment segments the region of interest R1 into a plurality of regions based on the fluid information on the part near the mitral valve in diastole. The processing after the region of interest R1 is segmented into a plurality of regions, that is, the processing at Steps S105 and S106 in FIG. 2 can be performed in the same manner as in the first embodiment, so explanation thereof is omitted.

While the embodiments above have described an example where the various types of fluid information are acquired based on the three-dimensional Doppler data, the embodiments are not limited thereto. For example, the processing circuitry 25 may acquire the fluid information based on MR images obtained by imaging the subject.

While the embodiments above have mainly described the mitral valve as the part of interest, the embodiments are not limited thereto. For example, the embodiments above are applicable to any desired living organ subjected to force due to the blood flow in part or the whole of the structure, such as the aortic valve, tricuspid valve, pulmonary artery valve, coronary artery, and left atrial appendage.

The embodiments above have described an example where the medical image I1 is acquired at Step S101 and the region of interest R1 is acquired based on the medical image I1. The embodiments, however, are not limited thereto. For example, an external device, such as the medical image diagnostic apparatus 10, may acquire the region of interest R1 from the medical image I1, and the processing circuitry 25 may acquire the region of interest R1 acquired by the external device via the network NW.

While the embodiments above have described the medical image diagnostic apparatus 10 and the medical image processing apparatus 20 as separate components, they may be integrated. For example, the medical image processing apparatus 20 may be a console included in the medical image diagnostic apparatus 10. In this case, the medical image processing apparatus 20 can acquire the medical image I1 by imaging the subject.

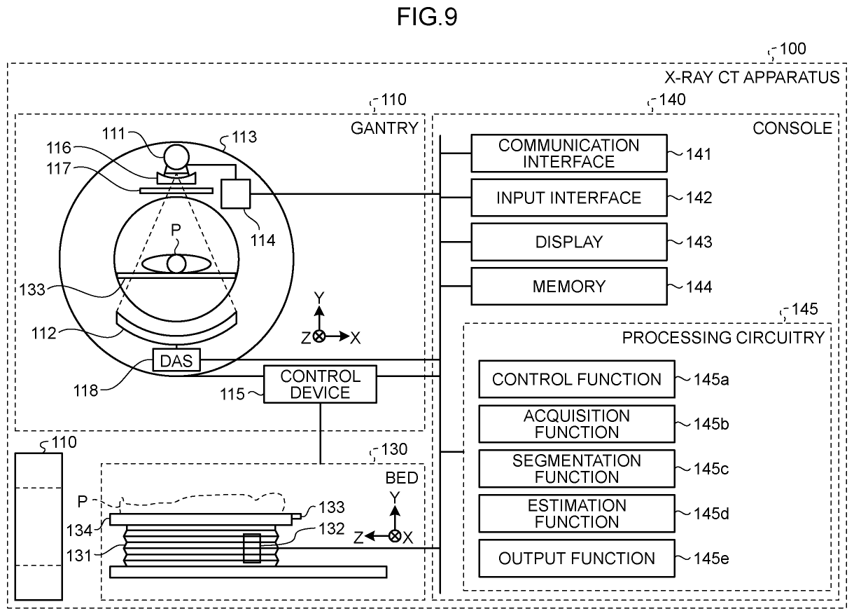

An X-ray CT apparatus 100 is illustrated in FIG. 9 as an example of the medical image diagnostic apparatus 10. The following describes an example where the same processing as that of the medical image processing apparatus 20 is performed in the X-ray CT apparatus 100. FIG. 9 is a block diagram of an example of the X-ray CT apparatus 100 according to another embodiment. The X-ray CT apparatus 100 includes, for example, a gantry 110, a bed 130, and a console 140.

In FIG. 9, a Z-axis direction is the direction of the rotation axis of a rotating frame 113 in a non-tilted state or the longitudinal direction of a tabletop 133 of the bed 130. The Z-axis direction corresponds to the body axis direction of the subject P placed on the tabletop 133. An X-axis direction is the axis direction orthogonal to the Z-axis direction and horizontal to the floor surface. A Y-axis direction is the axis direction orthogonal to the Z-axis direction and perpendicular to the floor surface. FIG. 9 illustrates the gantry 110 from a plurality of directions for explanation, and the X-ray CT apparatus 100 includes one gantry 110.

The gantry 110 includes an X-ray tube 111, an X-ray detector 112, the rotating frame 113, an X-ray high voltage device 114, a control device 115, a wedge 116, a collimator 117, and a data acquisition system (DAS) 118.

The X-ray tube 111 is a vacuum tube including a cathode (filament) that generates thermoelectrons and an anode (target) that generates X-rays due to the thermoelectrons colliding therewith. The X-ray tube 111 generates X-rays to be output to the subject P by outputting the thermoelectrons from the cathode to the anode due to the application of high voltage from the X-ray high voltage device 114. The X-ray tube 111 is an example of an X-ray generation unit.

The hardware that generates X-rays is not limited to the X-ray tube 111. For example, X-rays may be generated by a fifth-generation system instead of the X-ray tube 111. The fifth-generation system includes a focus coil that converges an electron beam generated from an electron gun, a deflection coil that electromagnetically deflects the electron beam, and a target ring that surrounds halfway around the subject P and generates X-rays by the deflected electron beam colliding therewith. In other words, the X-ray generator may be composed of a focus coil, a deflection coil, and a target ring.

The X-ray detector 112 detects X-rays output from the X-ray tube 111 and passing through the subject P and outputs signals corresponding to the detected X-ray dose to the DAS 118. The X-ray detector 112 includes, for example, a plurality of detection element rows each composed of a plurality of detection elements arrayed in a channel direction along one arc centered at the focus of the X-ray tube 111. In the X-ray detector 112, for example, a plurality of detection element rows each composed of a plurality of detection elements arrayed in the channel direction are arrayed in the column direction (slice direction or row direction). The X-ray detector 112 is an example of an X-ray detection unit.

The X-ray detector 112 is, for example, an indirect conversion detector including a grid, a scintillator array, and an optical sensor array. The scintillator array includes a plurality of scintillators. The scintillator includes a scintillator crystal that outputs light of a photon quantity corresponding to the incident X-ray dose. The grid is disposed on the surface of the scintillator array on which the X-rays are incident and includes an X-ray shielding plate that absorbs scattered X-rays. The grid may also be referred to as a collimator (one-dimensional collimator or two-dimensional collimator). The optical sensor array has a function to convert light from the scintillator into electrical signals corresponding to the light amount and includes, for example, optical sensors, such as photodiodes. The X-ray detector 112 may be a direct conversion detector including semiconductor elements that convert incident X-rays into electrical signals. Alternatively, the X-ray detector 112 may be a photon-counting detector that outputs signals with which the energy value of incident X-ray photons can be measured.

The rotating frame 113 is an annular frame that supports the X-ray tube 111 and the X-ray detector 112 in a manner facing each other and rotates the X-ray tube 111 and the X-ray detector 112 by the control device 115. The rotating frame 113 is, for example, a casting made of aluminum. The rotating frame 113 can further support the X-ray high voltage device 114, the wedge 116, the collimator 117, the DAS 118, and other components besides the X-ray tube 111 and the X-ray detector 112. The rotating frame 113 can further support various components not illustrated in FIG. 1. In the following description, the rotating frame 113 and the part that rotates and moves with the rotating frame 113 in the gantry 110 are also referred to as a rotating unit.

The X-ray high voltage device 114 includes an electric circuit, such as a transformer and a rectifier, and includes a high voltage generation device that generates high voltage to be applied to the X-ray tube 111 and an X-ray control device that controls the output voltage according to the X-rays generated by the X-ray tube 111. The high voltage generation device may have a transformer system or an inverter system. The X-ray high voltage device 114 may be provided to the rotating frame 113 or a fixed frame, which is not illustrated.

The control device 115 includes processing circuitry having a CPU or the like, and a drive mechanism, such as a motor and an actuator. The control device 115 receives input signals from an input interface 142 and controls the operations of the gantry 110 and the bed 130. The control device 115 controls, for example, the rotation of the rotating frame 113, the tilt of the gantry 110, and the movement of the bed 130. For example, the control device 115 rotates the rotating frame 113 around an axis parallel to the X-axis direction based on input inclination angle (tilt angle) information as the control to tilt the gantry 110. The control device 115 may be provided to the gantry 110 or the console 140.

The wedge 116 is an X-ray filter for adjusting the dose of X-rays output from the X-ray tube 111. Specifically, the wedge 116 is an X-ray filter that attenuates the X-rays output from the X-ray tube 111 such that the X-rays output from the X-ray tube 111 to the subject P have a predetermined distribution. The wedge 116 is, for example, a wedge filter or a bow-tie filter and is fabricated by processing aluminum or other material so as to have a predetermined target angle and a predetermined thickness.

The collimator 117 is a lead plate or the like to narrow the irradiation range of X-rays transmitted through the wedge 116 and forms a slit by combining a plurality of lead plates or the like. The collimator 117 may also be referred to as an X-ray diaphragm. While the wedge 116 is disposed between the X-ray tube 111 and the collimator 117 in FIG. 1, the collimator 117 may be disposed between the X-ray tube 111 and the wedge 116. In this case, the wedge 116 transmits and attenuates X-rays output from the X-ray tube 111 and having the irradiation range limited by the collimator 117.

The DAS 118 acquires the signals of X-rays detected by the detection elements included in the X-ray detector 112. For example, the DAS 118 includes an A/D converter that performs amplification on the electrical signals output from the detection elements to convert them into digital signals, and generates detection data. The DAS 118 is implemented by a processor, such as an application specific integrated circuit (ASIC).

The DAS 118 may have a simultaneous acquisition system or a sequential acquisition system. The DAS 118 with a simultaneous acquire system is provided for each detection element of the X-ray detector 112. The DAS 118 with a simultaneous acquisition system reads out a charge simultaneously when the charge is accumulated in the corresponding detection element, thereby generating detection data. The DAS 118 with a sequential acquisition system is provided for each set of detection elements (group of detection elements). The DAS 118 with a sequential acquisition system sequentially reads out the charges stored in the respective detection elements, thereby generating detection data.

The data generated by the DAS 118 is transmitted by optical communications from a transmitter provided to the rotating frame 113 and including light-emitting diodes (LEDs) to a receiver provided to a non-rotating part (e.g., fixed frame, which is not illustrated in FIG. 1) of the gantry 110 and including photodiodes, and is transferred to the console 140. The non-rotating part is, for example, a fixed frame that rotatably supports the rotating frame 113. The method for transmitting data from the rotating frame 113 to the non-rotating part of the gantry 110 is not limited to optical communications. Any non-contact data transmission method or a contact data transmission method may be employed.

The bed 130 is an apparatus for placing and moving the subject P to be scanned and includes a base 131, a bed drive device 132, the tabletop 133, and a support frame 134. The base 131 is a housing that supports the support frame 134 in a vertically movable manner. The bed drive device 132 is a drive mechanism that moves the tabletop 133 with the subject P placed thereon in the long-axis direction of the tabletop 133 and includes a motor, an actuator, and other components. The tabletop 133 provided on the upper surface of the support frame 134 is a board on which the subject P is placed. The bed drive device 132 may move the support frame 134 besides the tabletop 133 in the long-axis direction of the tabletop 133.

The console 140 includes a communication interface 141, an input interface 142, a display 143, a memory 144, and processing circuitry 145. While the console 140 is described as an apparatus separated from the gantry 110, the gantry 110 may include the console 140 or some of the components of the console 140.

The communication interface 141, the input interface 142, the display 143, and the memory 144 can be configured in the same manner as the communication interface 21, the input interface 22, the display 23, and the memory 24 described above, so detailed explanation thereof is omitted. The display 143 displays, for example, X-ray CT images and results of pressure estimation under the control of the processing circuitry 145.

The processing circuitry 145 implements a control function 145a, an acquisition function 145b, a segmentation function 145c, an estimation function 145d, and an output function 145e, thereby collectively controlling the operations of the X-ray CT apparatus 100. The control function 145a is an example of a control unit. The acquisition function 145b is an example of an acquisition unit. The segmentation function 145c is an example of a segmentation unit. The estimation function 145d is an example of an estimation unit. The output function 145e is an example of an output unit.

For example, the processing circuitry 145 functions as the control function 145a by reading a computer program corresponding to the control function 145a from the memory 144 and executing it. Similarly, the processing circuitry 145 can function as the acquisition function 145b, the segmentation function 145c, the estimation function 145d, and the output function 145e.

The control function 145a images the subject P and acquires X-ray CT images. For example, the control function 145a controls the X-ray high voltage device 114 to supply high voltage to the X-ray tube 111. Thus, the X-ray tube 111 generates X-rays to be output to the subject P. The control function 145a controls the bed drive device 132, thereby moving the subject P into the imaging opening of the gantry 110. The control function 145a adjusts the position of the wedge 116 and the aperture and position of the collimator 117, thereby controlling the distribution of X-rays output to the subject P. The control function 145a controls the control device 115, thereby rotating the rotating unit. While scanning is being performed by the control function 145a, the DAS 118 acquires the electrical signals output from the detection elements in the X-ray detector 112 and generates detection data.

The control function 145a performs preprocessing on the detection data output from the DAS 118. For example, the control function 145a performs preprocessing, such as logarithmic transformation, offset correction, sensitivity correction between channels, and beam hardening correction, on the detection data output from the DAS 118. The data resulting from preprocessing is also referred to as raw data. The detection data prior to preprocessing and the raw data resulting from preprocessing are also collectively referred to as projection data.

The control function 145a performs reconstruction based on the projection data to generate an X-ray CT image (volume data). The method of reconstruction is not particularly limited, and any desired method can be employed, such as filtered back projection and iterative reconstruction.

The acquisition function 145b is similar to the acquisition function 25a described above. For example, the acquisition function 145b acquires the region of interest based on the anatomical structure depicted in the X-ray CT image.

The segmentation function 145c is similar to the segmentation function 25b described above. For example, the segmentation function 145c acquires the fluid information indicating the blood flow related to the region of interest and segments the region of interest into a plurality of regions based on the fluid information.

The estimation function 145d is similar to the estimation function 25c described above. For example, the estimation function 145d estimates the pressure in the segmented regions.

The output function 145e is similar to the output function 25d described above. For example, the output function 145e performs output based on the results of pressure estimation.

In the X-ray CT apparatus 100 illustrated in FIG. 9, each processing function is stored in the memory 144 as a computer-executable program. The processing circuitry 145 is a processor that reads each computer program from the memory 144 and executes it to implement the function corresponding to the computer program. In other words, the processing circuitry 145 that has read a computer program has the function corresponding to the read computer program.

In FIG. 9, a single processing circuitry 145 implements the control function 145a, the acquisition function 145b, the segmentation function 145c, the estimation function 145d, and the output function 145e. Alternatively, a plurality of independent processors may be combined to constitute the processing circuitry 145, and each processor may execute the computer program to implement the corresponding function. Each processing function of the processing circuitry 145 may be distributed or integrated into a single or a plurality of processing circuits as appropriate. While the computer programs corresponding to the respective processing functions are stored in a single memory 144 in FIG. 9, the computer programs corresponding to the respective processing functions may be distributed and stored in a plurality of memories, and each computer program may be read from the corresponding memory and executed.

The processing circuitry 145 may implement the functions using a processor of an external apparatus connected via the network NW. For example, the processing circuitry 145 implements the functions illustrated in FIG. 1 by reading the computer programs corresponding to the respective functions from the memory 144 and executing them and using a group of servers (cloud) connected to the X-ray CT apparatus 100 via the network NW as computing resources.

The term “processor” used in the description above refers to, for example, a CPU, a graphics processing unit (GPU), or a circuit, such as an application specific integrated circuit (ASIC) and a programmable logic device (e.g., a simple programmable logic device (SPLD), a complex programmable logic device (CPLD), and a field programmable gate array (FPGA)). The processor reads and executes the computer programs stored in the memory to implement the functions.

In FIG. 1, a single memory 24 stores therein the computer programs corresponding to the respective processing functions. The embodiments, however, are not limited thereto. For example, a plurality of memories 24 may be distributed, and the processing circuitry 25 may read each computer program from the corresponding individual memory 24. Also in FIG. 9, a plurality of memories 144 may be distributed, and the processing circuitry 145 may read each computer program from the corresponding individual memory 144. Instead of being stored in the memory, the computer programs may be incorporated directly in the circuit of the processor. In this case, the processor reads and executes the computer programs incorporated in the circuit to implement the functions.

The components of the apparatuses according to the embodiments described above are functionally conceptual and are not necessarily physically configured as illustrated in the drawings. In other words, the specific forms of distribution and integration of the apparatuses are not limited to those illustrated in the drawings. All or part of the components may be functionally or physically distributed or integrated in desired units depending on various loads, states of use, and the like. All or desired part of the processing functions performed by the apparatuses are implemented by a CPU or a computer program analyzed and executed by the CPU or as hardware by wired logic.

The method described in the embodiments above can be performed by executing a computer program prepared in advance on a computer, such as a personal computer and a workstation. This computer program can be distributed via a network, such as the Internet. The computer program is recorded in a non-transitory computer readable medium, such as a hard disk, a flexible disk (FD), a CD-ROM, an MO, and a DVD, and can be executed by being read from the recording medium by a computer.

According to at least one of the embodiments described above, pressure in the parts of the cardiovascular system can be accurately estimated while reducing the processing load.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising processing circuitry configured to:
image a subject and acquire an X-ray CT image;
acquire a region of interest based on an anatomical structure depicted in the X-ray CT image;
acquire fluid information indicating a blood flow related to the region of interest and segment the region of interest into a plurality of regions based on the fluid information; and
estimate pressure in the plurality of regions.

2. A medical image processing apparatus comprising processing circuitry configured to:
acquire a region of interest based on an anatomical structure depicted in a medical image;
acquire fluid information indicating a blood flow related to the region of interest and segment the region of interest into a plurality of regions based on the fluid information; and
estimate pressure in the plurality of regions.

3. The medical image processing apparatus according to claim 2, wherein the processing circuitry is configured to acquire regurgitation velocity distribution in a neighborhood of the region of interest as the fluid information.

4. The medical image processing apparatus according to claim 2, wherein the processing circuitry is configured to acquire the fluid information based on three-dimensional Doppler data.

5. The medical image processing apparatus according to claim 2, wherein the processing circuitry is configured to acquire fluid information in systole of the heart as the fluid information.

6. The medical image processing apparatus according to claim 2, wherein the processing circuitry is configured to acquire fluid information in diastole of the heart as the fluid information.

7. The medical image processing apparatus according to claim 2, wherein the region of interest is a region corresponding to a part of interest subjected to force due to the blood flow.

8. The medical image processing apparatus according to claim 7, wherein the processing circuitry is further configured to estimate pressure distribution by modeling pressure applied to the part of interest based on the pressure estimated for each of the plurality of regions and performing parameter estimation.

9. The medical image processing apparatus according to claim 7, wherein
the part of interest is the mitral valve, and
the processing circuitry is configured to set the plurality of regions by segmenting the mitral valve between the farthest point of the anterior cusp valve annulus and the farthest point of the posterior cusp valve annulus at a specific ratio.

10. The medical image processing apparatus according to claim 2, wherein the processing circuitry is configured to set a region within a predetermined distance from the region of interest and acquire flow velocity information assigned to a pixel in the region as the fluid information.

11. The medical image processing apparatus according to claim 2, wherein the processing circuitry is configured to segment the region of interest into the plurality of regions by identifying a portion of the region of interest where a flow velocity value exceeds a predetermined threshold as a first segmented region and identifying a portion of the region of interest other than the first segmented region as a second segmented region.

12. The medical image processing apparatus according to claim 11, wherein the processing circuitry is configured to estimate pressure for each of the first segmented region and the second segmented region.

13. The medical image processing apparatus according to claim 11, wherein the processing circuitry is configured to identify a plurality of regions as the second segmented region.

14. The medical image processing apparatus according to claim 13, wherein
the processing circuitry is configured to identify a portion of the region of interest where the flow velocity value exceeds a first threshold as the first segmented region and identify a portion of the region of interest other than the first segmented region as the second segmented region, and
the processing circuitry is configured to identify a portion of the second segmented region where the flow velocity value exceeds a second threshold smaller than the first threshold as a first sub-segmented region and identify a portion of the second segmented region other than the first sub-segmented region as a second sub-segmented region.

15. The medical image processing apparatus according to claim 13, wherein the processing circuitry is configured to identify a portion of the second segmented region corresponding to the anterior cusp as a first sub-segmented region and identify a portion corresponding to the posterior cusp as a second sub-segmented region.

16. A method comprising:
acquiring a region of interest based on an anatomical structure depicted in a medical image;
acquiring fluid information indicating a blood flow related to the region of interest and segmenting the region of interest into a plurality of regions based on the fluid information; and
estimating pressure in the plurality of regions.

17. The method according to claim 16, wherein regurgitation velocity distribution in a neighborhood of the region of interest is acquired as the fluid information.

18. The method according to claim 16, wherein
the region of interest is a region corresponding to a part of interest subjected to force due to the blood flow,
the part of interest is the mitral valve, and
the plurality of regions are set by segmenting the mitral valve between the farthest point of the anterior cusp valve annulus and the farthest point of the posterior cusp valve annulus at a specific ratio.

19. The method according to claim 16, wherein the region of interest is segmented into the plurality of regions by identifying a portion of the region of interest where a flow velocity value exceeds a predetermined threshold as a first segmented region and identifying a portion of the region of interest other than the first segmented region as a second segmented region.

20. A non-transitory computer readable medium comprising instructions that cause a computer to execute:
acquiring a region of interest based on an anatomical structure depicted in a medical image;
acquiring fluid information indicating a blood flow related to the region of interest and segmenting the region of interest into a plurality of regions based on the fluid information; and
estimating pressure in the plurality of regions.
